(19) United States
(12) Patent Application Publication
(10) Pub. No.: US 2018/0092791 A1
(43) Pub. Date: Apr. 5, 2018

(54) Removable Joint Sleeve

(71) Applicant: Gregg Alper, Delray Beach, FL (US)

(72) Inventor: Gregg Alper, Delray Beach, FL (US)

(21) Appl. No.: 15/332,030

(22) Filed: Oct. 24, 2016

Related U.S. Application Data
(60) Provisional application No. 62/403,239, filed on Oct. 3, 2016.

(51) Int. Cl. A61H 1/00; A41D 13/06; A41D 13/08; A61F 5/30

(52) U.S. Cl. CPC A61H 1/008; A41D 13/065; A61H 2205/102; A61F 5/30; A61H 2205/06; A41D 13/08

(57) ABSTRACT

A removable joint sleeve. The sleeve comprises a sleeve for joints. The sleeve comprises an inner layer, outer layer, upper hem, lower hem, internal flap, zipper and zipper tab, zipper trim, and zipper guard. The sleeve uses a zipper primarily due to the ease with which it can be opened and closed. Preferably, the zipper guard is a hook and loop connection like Velcro® or other similar materials. The zipper guard is preferably rounded so as to prevent fraying and prevents the zipper from unzipping during adjustments or extreme movements. The zipper guard shape also prevents the zipper guard from rolling up or folding. The internal flap sits between the zipper and user's skin to prevent any snagging of hair or skin. The zipper trim creates a zipper valley as well as a pleasing aesthetic and prevents fraying or unraveling. Preferably, the sleeve is made of neoprene or a similar material so as to stretch around the user's appendage and compress around it, providing therapeutic pressure.

### RELATED APPLICATION DATA

This application claims priority to Provisional Application No. 62/403,239 filed on Oct. 3, 2016 and entitled “Removable Joint Sleeve.” The content of this application is fully incorporated herein for all purposes.

### TECHNICAL FIELD

The present invention relates to a sleeve. More specifically, the present invention relates to a removable sleeve which wraps around a user's joint generally during athletic activity to provide therapeutic compression.

### BACKGROUND OF THE INVENTION

For athletes who participate in strenuous workouts, whether that is power lifting, running, or a combination of the two, a large amount of pressure is put on the knees and other joints. Those athletes with weaker joints generally need a form of support. Previously, supports for weak joints have existed and have helped to provide support by providing compression around the joint and retaining heat and were generally constructed from neoprene or other similarly stretchable synthetic fabric.

However, existing joint sleeves require the user to have a clear area with which to apply and remove the sleeve. As an example, knee sleeves require the user to remove their shoes before donning or doffing the sleeve. This can result in a delayed or stilted workout as some exercises may require the sleeve while others may be impeded by the sleeve if left on the joint.

There have been attempts to make removable joint sleeves. For example, U.S. Pat. No. 8,118,762 to Bort discloses a removable medical knee bandage using a zipper. However, this disclosure does not include any sort of means for preventing the zipper from disengaging while the sleeve is in use that is easy to undo or put into place. It uses a button system to keep the zipper in place which can be difficult to implement with sweaty fingers and can easily fray due to its shape. The present invention includes an easy to engage and disengage hook and loop flap which features a rounded design which eliminates the possibility of the material curling up or folding, which would normally occur with a square flap. Additionally, while Bort includes an interior structure separating the user's skin from the sleeve, the interior structure is sewn to the supporting edges on both sides and extends across the entire length of the separation. The present invention includes interior and exterior edges, trimmed with the same material, in order to prevent chafing and discomfort. The interior edges do not extend across the entire length of the sleeve because it would otherwise cause the material to fold over itself during the zippering process. The trim also helps to prevent the material from unraveling or fraying.

Therefore, it is an object of this invention to provide an improvement which overcomes the aforementioned inadequacies of the prior art devices and methods and provides an improvement which is a significant contribution to the advancement of joint sleeve art.

It is a further object of this invention to provide a removable joint sleeve made of neoprene or other similarly stretchable material.

It is a further object of this invention to provide a removable joint sleeve which will not fray or unravel.

It is a further object of this invention to provide a removable joint sleeve which can be used during high intensity athletic activities to provide support, compression, and heat retention.

It is a further object of this invention to provide a removable joint sleeve which does not require the removal of outerwear to don or doff.

The foregoing has outlined some of the pertinent objects of this invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

For the purpose of summarizing this invention, this invention comprises a sleeve for joints. The sleeve comprises an inner layer, outer layer, upper hem, lower hem, internal flap, zipper and zipper tab, zipper trim, and zipper guard. The sleeve uses a zipper primarily due to the ease with which it can be opened and closed. Preferably, the zipper guard is a hook and loop connection like Velcro® or other similar materials. The zipper guard is preferably rounded so as to prevent fraying and prevents the zipper from unzipping during adjustments or extreme movements. The zipper guard shape also prevents the zipper guard from rolling up or folding. The internal flap sits between the zipper and user's skin to prevent any snagging of hair or skin. The zipper trim creates a zipper valley as well as a pleasing aesthetic and prevents fraying or unraveling. Preferably, the sleeve is made of neoprene or a similar material so as to stretch around the user's appendage and compress around it, providing therapeutic pressure.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiments disclosed may be readily utilized as a basis for modifying or designing other structures and methods for carrying out the same purposes of the present invention. It should also be realized by those skilled in the art that such equivalent constructions and methods do not depart from the spirit and scope of the invention as set forth in the appended claims.

### BRIEF DESCRIPTION OF THE DRAWINGS

For a fuller understanding of the nature and objects of the invention, reference should be had to the following detailed description taken in connection with the accompanying drawings in which:

FIG. 1A illustrates the removable joint sleeve in place around a user's arm.

FIG. 1B illustrates the removable joint sleeve in place around a user's leg.

FIG. 2 is a front perspective view of the removable joint sleeve with the zipper closed and locked into place with the zipper guard.

FIG. 3 is a rear perspective view of the removable joint sleeve.

FIG. 4 is a side perspective view of the removable joint sleeve.

FIG. 5 is another side perspective view of the removable joint sleeve.

FIG. 6 is a perspective view of the inside of the removable joint sleeve unzipped.

FIG. 7 is a perspective view of the outside of the removable joint sleeve unzipped.

Similar reference characters refer to similar parts throughout the several views of the drawings.

### DETAILED DESCRIPTION OF THE PREFERRED EMBODIMENT

The following description is of a preferred embodiment presently contemplated for carrying out the invention. This description is not to be taken in a limiting sense, but is made merely for the purpose of describing one or more preferred embodiments of the invention. The scope of the invention should be determined with reference to the claims.

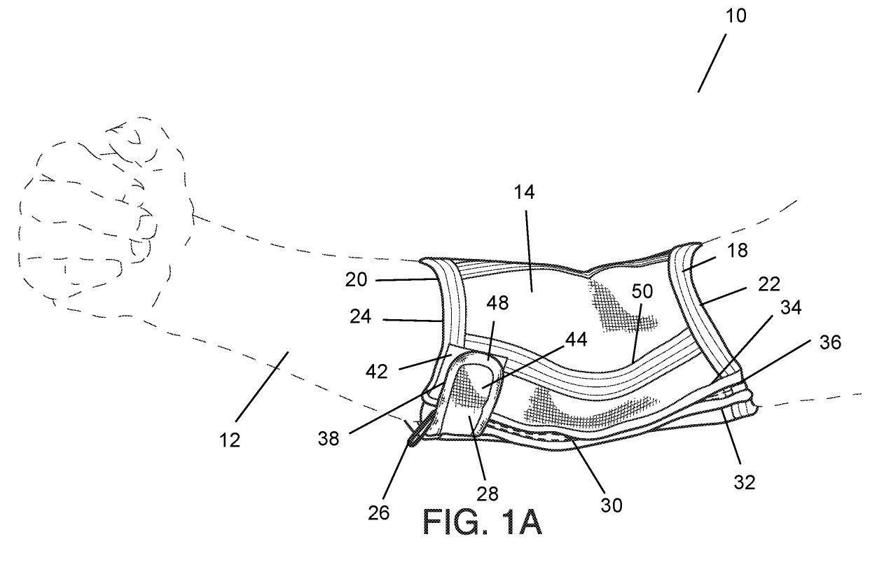

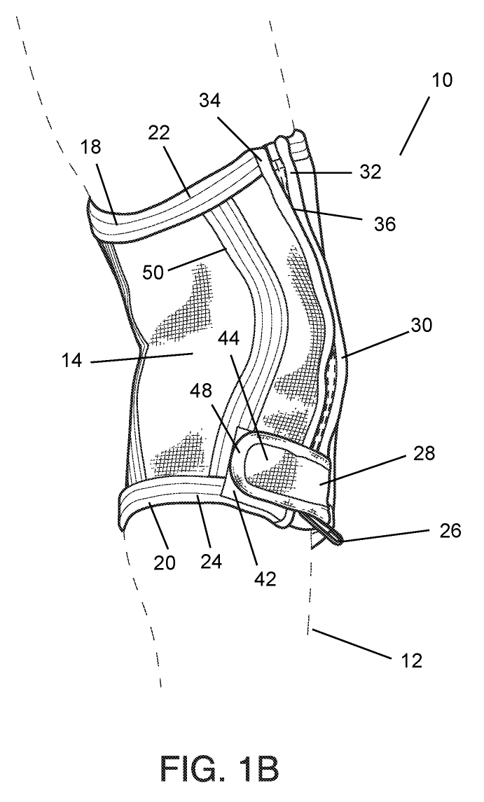

FIG. 1A depicts the removable joint sleeve 10. In this embodiment, the sleeve is wrapped around a user's appendage 12 which in this embodiment is an arm but can be any appendage. The removable joint sleeve 10 comprises a first side 14 and second side 16. The removable joint sleeve 10 is a single continuous piece of material and is preferably neoprene or some other similarly stretchable material. The removable joint sleeve 10 further comprises an upper trim 18 and lower trim 20 which is substantially continuous around the upper edge 22 and lower edge 24 of the removable joint sleeve. The upper trim 18 and lower trim 20 serve to provide comfort against the user's skin and to prevent fraying and/or unraveling. The upper trim 18 and lower trim 20 are preferably 2-3 inches in length to maximize this effect.

FIG. 1B depicts an embodiment wherein appendage 12 is a leg. This view allows for a better view of the zipper 26, zipper guard 28, and zipper trim 30. The zipper trim 30 is at the first end 32 and second end 34 of the removable joint sleeve 10. The zipper trim 30 recesses the zipper 26 into the removable joint sleeve 10 protecting the zipper 26 from snagging onto anything while moving around. The zipper trim 30 also allows the removable joint sleeve 10 to be zipped up or down using a single hand without having material fold over due to the finished edge 36 which also serves to prevent unraveling or fraying.

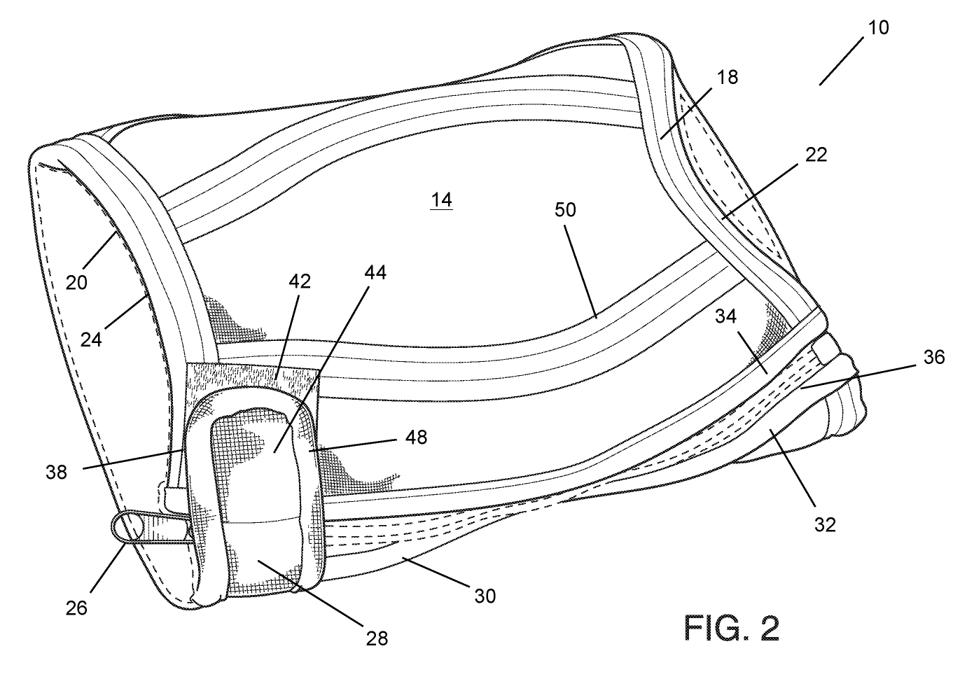

FIG. 2 is a perspective view of the removable joint sleeve 10. The zipper guard 28 is preferably made of the same material as the removable joint sleeve 10. The removable joint sleeve uses a hook and loop connection 38 using a hook pad 40 and loop pad 42. The zipper guard 28 has a first guard side 44 and second guard side 46. The second guard side 46 comprises the hook pad 40. The loop pad 42 is placed on the removable joint sleeve 10 in a position such that the zipper 26 is locked into place when closed. The zipper guard 28 also has zipper guard trim 48 to prevent fraying or unraveling. The zipper guard 28 prevents unzipping during extreme movement or during adjustment on the appendage. Preferably, the zipper guard 28 is rounded so as to eliminate curling or folding of material which would normally occur with a square shaped closure.

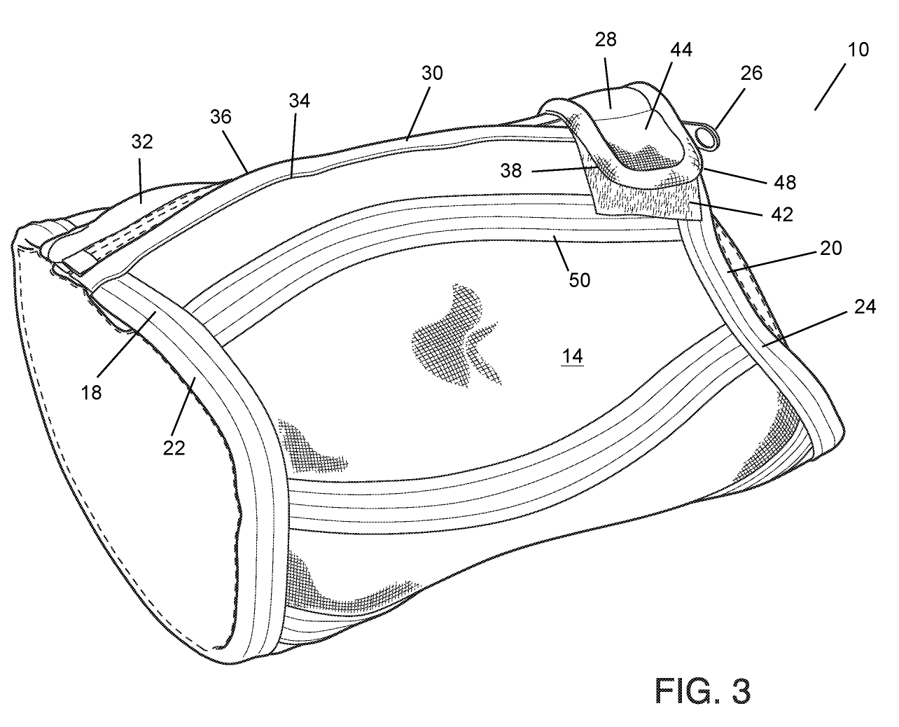

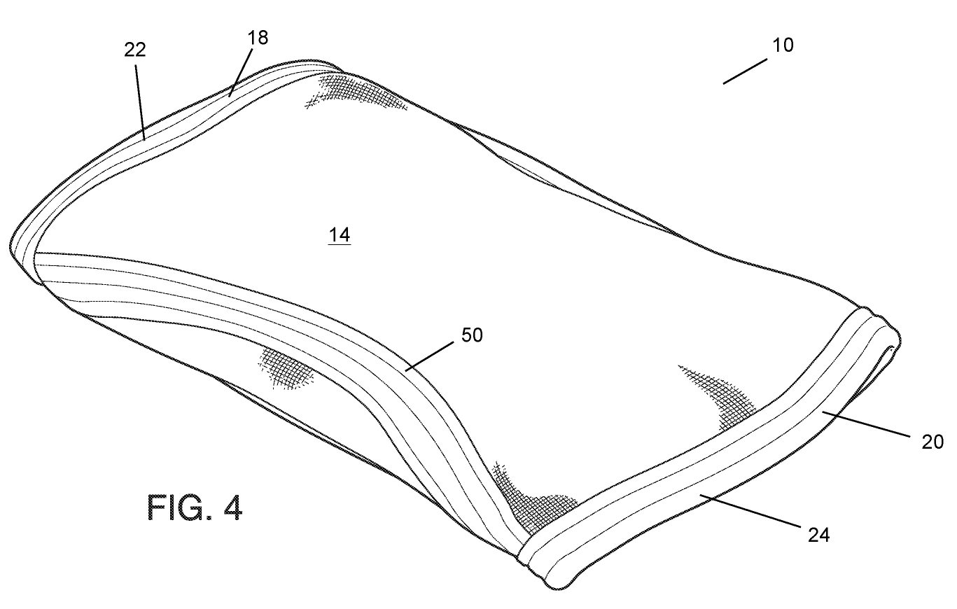

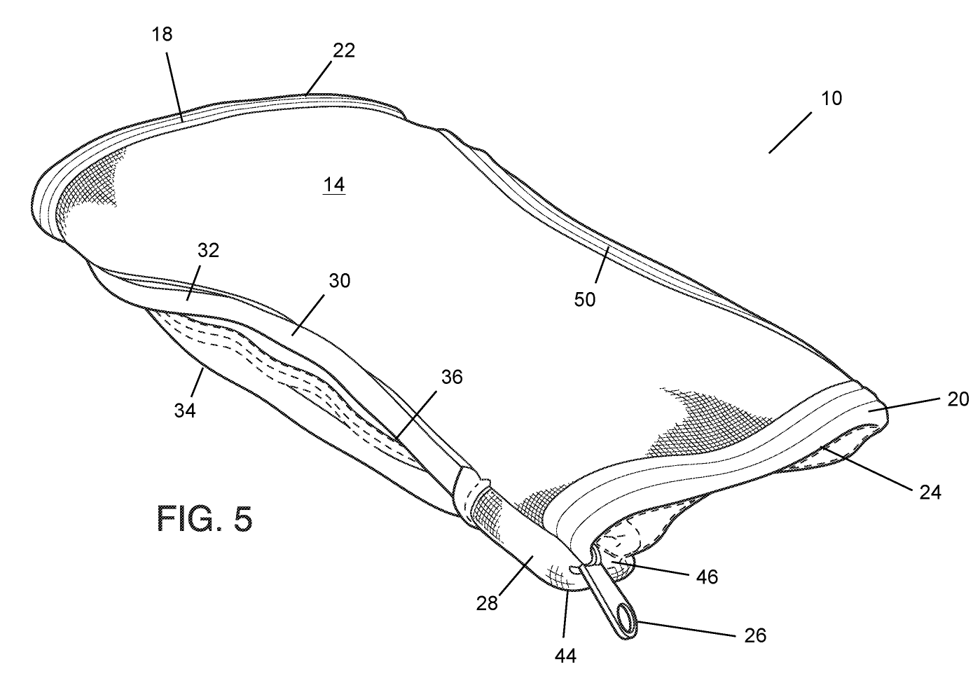

FIGS. 3, 4, and 5 show different perspective views of the removable joint sleeve 10. As can be seen in these views, the removable joint sleeve 10 has a plurality of support strips 50. These support strips 50 provide further support and compression for therapeutic comfort while exercising.

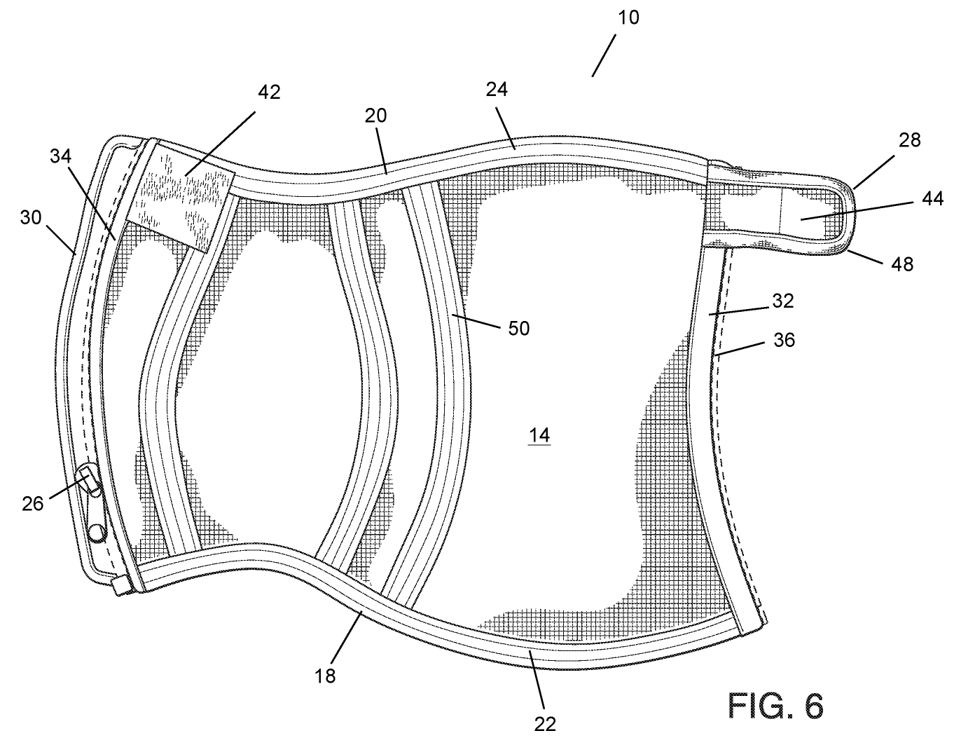

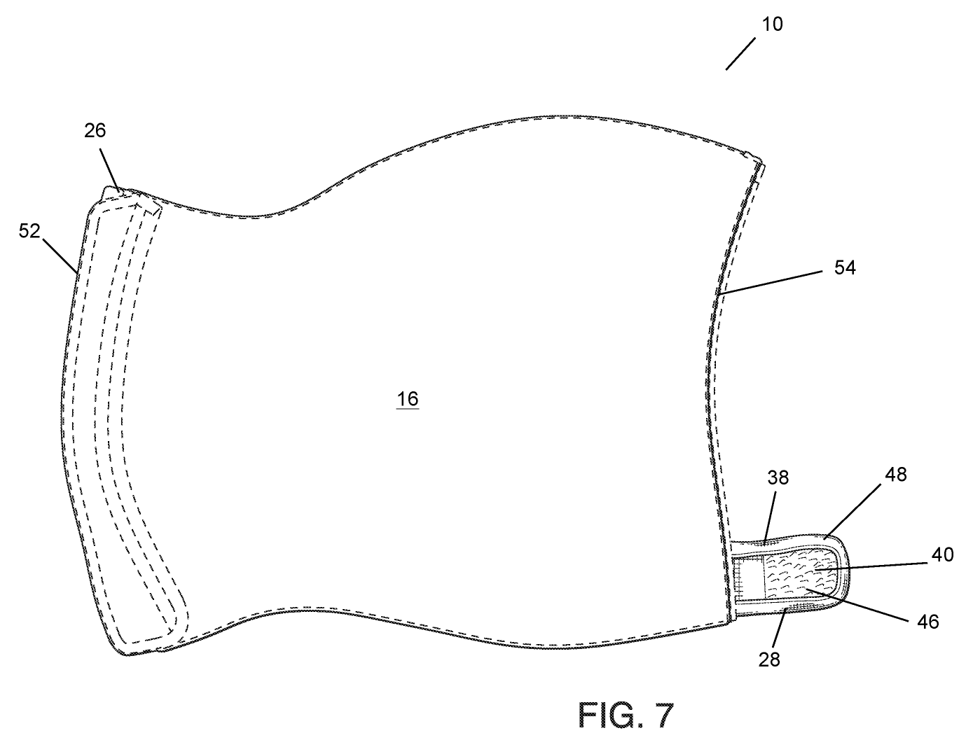

FIGS. 6 and 7 provide an exterior and interior view of the removable joint sleeve 10 when unzipped. The removable joint sleeve 10 further comprises a first interior flap 52 and second interior flap 54. Each flap sits between the zipper 26 and the user's skin and provide a barrier when the zipper 26 is closed. The support strips 50 can be seen on both the first side 14 and second side 16 and extend the length of the removable joint sleeve 10. As can be seen in FIG. 6, the loop pad 42 is attached to the second end 34. FIG. 7 shows an alternative embodiment where support strips 50 are not visible on the second side 16 of the removable joint sleeve 10.

The invention being described, it will be obvious that the same may be varied in many ways. Such variations are not to be regarded as a departure from the spirit and scope of the invention, and all such modifications as would be obvious to one of ordinary skill in the art are intended to be included within the scope of the following claims.

What is claimed is:

1. A removable joint sleeve comprising:
a sleeve comprising a first sleeve side and a second sleeve side made of stretchable material further comprising an upper edge, a lower edge, a first sleeve end, and a second sleeve end;
an upper trim covering the upper edge and a lower trim covering the lower edge;
a zipper system comprising a zipper having a first zipper side and second zipper side, zipper guard, and zipper trim wherein the first zipper side is attached to the first sleeve end and the second zipper side is attached to the second sleeve end and wherein the zipper guard comprises a first guard side and second guard side wherein the second guard side comprises a hook pad for a hook and loop connection;
a loop pad placed on the first sleeve side such that it is in contact with the hook pad when placed over the zipper;
at least one support strip stitched into the sleeve.

2. The removable joint sleeve of claim 1, wherein the stretchable material is neoprene.

3. The removable joint sleeve of claim 1, wherein the removable joint sleeve conforms to the shape of a user's appendage joint when the zipper is closed.

4. The removable joint sleeve of claim 3, wherein the user's appendage joint is a knee.

5. The removable joint sleeve of claim 3, wherein the user's appendage joint is an elbow.

6. The removable joint sleeve of claim 1, wherein the removable joint sleeve therapeutically applies pressure to a user's appendage joint when wrapped around said appendage.

7. A removable joint sleeve comprising:
a sleeve comprised of stretchable material further comprising a first sleeve end and second sleeve end;
a zipper system comprising a zipper having a first zipper side and second zipper side, wherein the first zipper side is attached to the first sleeve end and the second zipper side is attached to the second sleeve end;
a zipper guard comprising a first guard side and second guard side wherein the second guard side comprises a hook pad for a hook and loop connection;
a loop pad placed on the first sleeve side such that it is in contact with the hook pad when placed over the zipper;
at least one support strip stitched into the sleeve.

8. The removable joint sleeve of claim 7, wherein the stretchable material is neoprene.

9. The removable joint sleeve of claim 7, wherein the removable joint sleeve conforms to the shape of a user's appendage joint when the zipper is closed.

10. The removable joint sleeve of claim 9, wherein the user's appendage joint is a knee.

11. The removable joint sleeve of claim 9, wherein the user's appendage joint is an elbow.

12. The removable joint sleeve of claim 7, wherein the removable joint sleeve therapeutically applies pressure to a user's appendage joint when wrapped around said appendage.
